(19) United States
(12) Patent Application Publication
(10) Pub. No.: US 2005/0209787 A1
(43) Pub. Date: Sep. 22, 2005

(54) Sequencing data analysis

(76) Inventors: Thomas B. Waggener, Newton, MA (US); Joseph A. Majzoub, Wellesley, MA (US)

Correspondence Address: FISH & RICHARDSON PC, P.O. BOX 1022, MINNEAPOLIS, MN, 55440-1022, US

(21) Appl. No.: 11/009,100

(22) Filed: Dec. 10, 2004

Related U.S. Application Data
(60) Provisional application No. 60/529,274, filed on Dec. 12, 2003.
(60) Provisional application No. 60/550,784, filed on Mar. 5, 2004.
(60) Provisional application No. 60/591,668, filed on Jul. 28, 2004.

(51) Int. Cl. G06F 19/00; G01N 33/48; G01N 33/50

(52) U.S. Cl. 702/20

(57) ABSTRACT

Sequence data is analyzed using one or more parameters; and a particular amplicon can be organized according to whether further review by a technician is needed. Sequence data can also be processed to identify performance alterations in a sequencing apparatus.

### CROSS-REFERENCE TO RELATED APPLICATIONS

This application claims priority to U.S. Application Ser. No. 60/529,274, filed on 12 Dec. 2003, Ser. No. 60/550,784, filed Mar. 5, 2004, and Ser. No. 60/591,668, filed on 28 Jul. 2004, the contents of each of which are hereby incorporated by reference in their entireties.

### BACKGROUND

When a DNA amplicon is sequenced to identify variations from a reference sequence, standard laboratory practice typically includes inspection of the data from sequencing of every amplicon by a technician for base calling accuracy and for variants. This process can be time consuming and expensive. An amplicon is a physical DNA fragment which typically includes a target region for sequencing. As used herein, “amplicon” may also refer to the sequence data obtained from analysis of the DNA fragment. An “amplicon” need not be a piece of DNA that has been amplified, but can refer to any DNA which is analyzed.

There is a need to reduce human technician time spent on producing and evaluating nucleic acid sequence information.

### SUMMARY

This disclosure includes, inter alia, a number of methods that can be used to process sequence data obtained from nucleic acid sequencing. Sequence data includes any form of raw and/or processed data obtained from monitoring a sequencing reaction, e.g., data from a sequencing apparatus such as an automated capillary electrophoresis sequencer. Examples of sequence data include “base calls” or nucleotide assignments, quality values, amplitudes, and peak widths.

The methods can be implemented using computer systems and can improve the efficiency of handling sequencing projects. These methods can also reduce the time required from human operators to oversee sequencing projects. The disclosure includes methods for screening and categorizing amplicon data so as to reduce the technician workload and methods for monitoring and evaluating DNA sequencer function.

In one aspect, the disclosure features a method of processing sequence data. The method includes: obtaining sequence data that includes nucleotide assignments for positions in a sequence and performance characteristics; and automatically sorting the sequence data into categories based on necessity for further review of the correctness of the sequence, e.g., manual review. Exemplary performance characteristics include quality value scores, amplitudes and/or peak widths for positions in the sequence.

The categories can include, for example, (i) one or more categories for sequence data that do not require further review of the correctness of the sequence, e.g., manual review; and (ii) one or more categories for sequence data that require further review of the correctness of the sequence, e.g., manual review. The method can further include providing the sequence data to an end user, e.g., a healthcare provider of the subject who provided the sequence.

The categories (i) of sequence data that do not require further review of the correctness of the sequence, e.g., manual review, can include a category for sequence data that includes accepted performance characteristics (e.g., at all or a threshold number or percentage of positions) and nucleotide assignments that match a reference sequence (e.g., at all or a threshold number or percentage of positions). For example, this category can be for “normal” sequence data. The method can include associating an identifier that indicates there is no need for resequencing. The method can further include: providing the sequence data to an end user, e.g., a healthcare provider providing healthcare to the subject which provided the sequence.

The categories (i) of sequence data that do not require further review of the correctness of the sequence, e.g., manual review can include a category for sequence data that includes a threshold number of unaccepted performance characteristics and at least a threshold number of nucleotide assignments that do not match a reference sequence. The method can include associating an identifier which indicates the need for resequencing. This sequence data can be indicated as “bad” and an instruction can be generated for automatically resequencing.

The categories (i) of sequence data that do not require further review of the correctness of the sequence, e.g., manual review, can include a category for sequence data that includes at least one unaccepted performance characteristic at a position, which characteristic is predicted to occur within the context of the position. (accepted based on signature). It is possible to associate an identifier which indicates there is no need for resequencing.

The categories (ii) of sequence data that do require further review of the correctness of the sequence, e.g., manual review can include a category for sequence data that includes at least a threshold number of nucleotide assignments that do not match a reference sequence and a threshold number of accepted performance characteristics (“IN/DELS”).

The categories (ii) of sequence data that do require further review of the correctness of the sequence, e.g., manual review, include a category for sequence data that includes a nucleotide assignment that does not match a reference sequence and an accepted performance characteristic at the position corresponding to the mismatch. (“variants”). It is possible to associate an identifier which indicates there is a need for review of the sequence.

The sequence data can be pre-processed, e.g., by software that determines nucleotide assignments (“base calls”) and other characteristics, e.g., quality values.

In one embodiment, the sequence data is trimmed to remove non target, e.g., terminal regions, e.g., so that the sequence data corresponds to only a portion of the amplicon.

In one embodiment, multiple files for sequence data are handled, and the files are organized by the automatic sorting. For example, the files are put into folders according to category, are indexed according to category, or are assigned an indicator according to category. It is possible to alert an operator of files in categories for samples that require review. For example, the operator is altered by a sequence of windows, each window including information for the operator to review (“pop up windows”).

The method can further include storing information about events, e.g., events associated with file reviews and categorization, e.g., by logging events, e.g., manual edits.

In another aspect, the disclosure features a method of processing sequence data, The method includes: obtaining sequence data that includes nucleotide assignments for positions in a sequence and performance characteristics; and evaluating the sequence data by determining one or more of the following: (i) if the sequence data includes accepted performance characteristics and nucleotide assignments that match a reference sequence (e.g., “normal”); (ii) if the sequence data includes a threshold number of unaccepted performance characteristics and at least a threshold number of nucleotide assignments that do not match a reference sequence (“bad”, e.g., indicate as automatically resequence); (iii) if the sequence data includes at least one unaccepted performance characteristic at a position, which characteristic is predicted to occur within the context of the position (e.g., accepted based on signature); (iv) if the sequence data includes at least one unaccepted performance characteristic at a position, which characteristic is accepted based on a revised quality value score; (v) if the sequence data includes at least one unaccepted performance characteristic at a position and nucleotide assignments that match a reference sequence (e.g., “low quality value score” class); (vi) if the sequence data includes at least a threshold number of nucleotide assignments that do not match a reference sequence and a threshold number of accepted performance characteristics (“IN/DELS”); and/or (vii) if the sequence data includes a nucleotide assignment that does not match a reference sequence and an accepted performance characteristic at the position corresponding to the mismatch (e.g., variants).

In one embodiment, item (iv) is determined using a Bayesian inference. For example, the inference is determined using two populations, e.g., one which includes matched positions, and one which includes unmatched positions, or populations based on whether the base call occurs in the same region of the amplicon as a reference sequence.

In one embodiment, the sequence data is evaluated for at least two, three, or four of the seven characteristics of (i)—(vii). For example, the sequence data is evaluated for at least all seven characteristics of (i)—(vii). In one embodiment, the sequence data is indicated for operator review if it has characteristic (v), (vi) or (vii).

The evaluating can be performed by a computational device, e.g., a microprocessor, a computer or other device. The method can include other features described herein.

In another aspect, the disclosure features a dataserver including storage (e.g., memory) having encoded therein multiple files of sequence data that includes nucleotide assignments for positions in a sequence and performance characteristics, wherein the files are organized according to one or more of the following categories, in which the sequence data: (i) includes accepted performance characteristics and nucleotide assignments that match a reference sequence (“normal”); (ii) includes a threshold number of unaccepted performance characteristics and at least a threshold number of nucleotide assignments that do not match a reference sequence (“bad”—automatically resequence); (iii) includes at least one unaccepted performance characteristic at a position, which characteristic is predicted to occur within the context of the position (accepted based on signature); (iv) includes at least one unaccepted performance characteristic at a position, which characteristic is accepted based on a revised quality value score; (v) if the sequence data includes at least one unaccepted performance characteristic at a position and nucleotide assignments that match a reference sequence (“low quality value score”); (vi) includes at least a threshold number of nucleotide assignments that do not match a reference sequence and a threshold number of accepted performance characteristics (“IN/DELS”); and/or (vii) includes a nucleotide assignment that does not match a reference sequence and an accepted performance characteristic at the position corresponding to the mismatch (“variants”). The dataserver can include other features described herein.

In another aspect, the disclosure features a method of identify insert/deletions (IN/DEL) in sequence data. The method includes: obtaining sequence data that includes nucleotide assignments for positions in a sequence and performance characteristics; and evaluating if the sequence data includes at least a threshold number of nucleotide assignments that do not match a reference sequence and a threshold number of accepted performance characteristics. Many IN/DELS are heterozygous. Fixing the IN/DEL includes more than shifting the sequence. In such cases, the method can further include adding or subtracting signals expected for a normal sequence from a region that includes mismatches to the reference sequence, and determining if the remaining signal corresponds to the reference sequence shifted by one or more positions. It is possible resolve the heterozygous calls relative to the reference sequence and then shift the unresolved half of the signal. Homogzygous IN/DELS can be resolved by simple shifting.

In another aspect, the disclosure features a method for evaluating sequence data, for example, output from a sequencer, e.g., an automated sequencer. The method includes: identifying at least one position in a sequence that has an unaccepted performance characteristic; and determining if the unaccepted performance is predicted to occur within the context of the position. In one embodiment, the method also includes if the unacceptable performance is predicted to occur within the context, then accepting the base call for the sequence and/or, if the unacceptable performance is not predicted to occur within the context, then not accepting the base call for the sequence.

In one embodiment, the step of determining includes accessing a database that includes records that associates performance characteristics (e.g., quality value scores) and sequence information, e.g. strings of nucleotides, e.g., strings corresponding to less than 9, 8, 7, 6, or 5 nucleotide positions. In one embodiment, the database includes records for each of at least a certain percentage of (e.g., 10, 20, 30, 40, 50, 80, 90, or 95) or all possible 3-mer, 4-mers, or 5-mers. For example, the database includes records for at least 10% of all possible 4-mers.

The database can be generated by evaluating sequence data produced from different samples (e.g., at least 2, 5, 20, 200, 500, 1000, or 5000), and recurring patterns of performance characteristics associated with a particular context of nucleotides are stored in the database. The database can be keyed, e.g., to a position at which an altered performance characteristic recurs.

The method can further include indicating the sequence data as accepted if the unaccepted performance is predicted to occur within the context of the position. For example, the unaccepted performance includes a quality value less than a threshold. The method can include other features described herein.

In another aspect, the disclosure features a method for evaluating sequence data, for example, output from a sequencer, e.g., an automated sequencer. The method includes: providing a database which includes sequences and sets of values associated with the respective sequences, the values being a value for a performance characteristic); and locating at least one position in a sequence, which is a position subject question, (e.g., a position characterized by a low quality score) and at least one additional position (e.g., at least one, two, or three adjacent positions); and determining if the nucleotide assignment for a position and the at least one additional position of a set of positions and their corresponding values match a record in the database.

The method can further include providing an indication that sequence data should be retained, e.g., not flagged for further analysis, if a match is detected. The method include other features described herein.

In another aspect, the disclosure features a method for evaluating sequence data, for example, output from a sequencer, e.g., an automated sequencer. The method includes: receiving sequence data that includes nucleotide assignments for positions in a sequence and values for a parameter that characterizes each position; evaluating the sequence data to identify a position, if any, for which the value is indicated as deviating from normal; comparing a pattern of values at consecutive positions, one of which is the identified position, to a database that associates patterns of values with strings of nucleotide assignments; and indicating the sequence data as accepted if the pattern of values for the consecutive positions is indicated by the database as associated with the nucleotide assignments for the consecutive positions. The method can include other features described herein.

In another aspect, the disclosure features a computer database that stores records that associates performance characteristics for a string of nucleotide assignments, e.g., a string corresponding to less than 9, 8, 7, 6, or 5 nucleotide positions. In one embodiment, the database includes records for each of all possible 3-mer, 4-mers, or 5-mers.

The database can be generated by evaluating at sequence data produced from at least different samples (e.g., at least 5, 20, 50, 100, 1000), and recurring patterns of performance characteristics associated with a particular context of nucleotides are stored in the database. Exemplary performance characteristics include quality values, scaled amplitudes, peak widths, or amplitude/peak width ratios, and values that are functions of these characteristics.

In another aspect, the disclosure features a method for evaluating the performance quality of one or more datasources for nucleic acid sequence data. The method includes: providing values for one or more parameters obtained from sequence data output from multiple datasources, organizing the parameter values according to datasource, and identifying, from the organized parameters, an indication of performance quality of one or more of the datasources or a component associated with the datasources.

In one embodiment, the multiple datasources correspond to reaction chambers or parallel tracks in a nucleic acid sequence apparatus, e.g., capillaries located in parallel in an automated nucleic acid sequencer. In one embodiment, the multiple datasources include datasources from different apparati.

In one embodiment, the step of organizing and/or identifying includes organizing the parameters as a data structure including two dimensions. In one embodiment, the data structure corresponds to a plate map.

In one embodiment, the step of organizing and/or identifying includes displaying information in a two dimensional grid, wherein parameters obtained from the same datasource are represented at positions along a line on one of the dimensions of the grid.

For example, the parameters are represented by colors from a color scale. In another example, the parameters are represented by a graph along a third dimension.

In one embodiment, the step of organizing and/or identifying includes detecting patterns indicative of reduced performance of one or more of the datasources. Detection of a pattern indicative of reduced performance can trigger an alert to a user, e.g., a flag that arrests the sequencer from processing another plate or sample. The method can include other features described herein.

In another aspect, the disclosure features a method for evaluating the performance quality of one or more components of an automated nucleic acid sequencing apparatus. The method includes: receiving values for one or more parameters obtained from sequence data output from multiple datasources, each datasource corresponding to a capillary of the apparatus, organizing the parameter values in an at least two-dimensional array wherein parameters from the same datasource are arranged in a linear series along one dimension of the array, and identifying, if present, a pattern of altered performance associated with one or more of the series, thereby generating an indication of performance quality of one or more of the datasources or components associated with the datasources. The method can include other features described herein.

In another aspect, the disclosure features a method that includes calculating quality value scores using two populations of base calls. In one embodiment, the base calls can be compared to a reference sequence. Base calls can be separated into two populations, those which match the reference sequence and those which do not. Methods disclosed herein can consider these two populations separately to determine quality value scores. In another embodiment, the two populations are based on whether the base call occurs in the same region of the amplicon as a reference sequence (e.g., a population of base calls within the same region, and a population of base calls that are outside the region). The method can include additional features described herein.

The disclosure also includes methods for monitoring events associated with editing and potential editing. For example, it is possible to generate an event file during screening and to use the event file to step through all potential edits. The user does not have to separately load and review amplicon data. For example, each event potentially needing an edit can be presented to the user in separate windows, e.g., windows that pop up sequentially.

In one aspect, the disclosure includes a method that calculates a posterior probability for each base call based on prior probabilities. The method provides new quality value scores, and is not dependent on a separate or new evaluation of the trace.

The methods described herein include ones that improve the accuracy of the calculation of the probability of error in a given base call. Information from the processing and analysis of both the raw electropherogram and the processed electropherogram can be used to classify the amplicons and/or sequence data from the amplicons. The methods can be implemented using a variety of software and/or hardware tools, e.g., a screening tool and in a sequencer function tracking tool.

This application incorporates all patents, applications, and references referenced herein, including U.S. Application Ser. No. 60/529,274, filed on 12 Dec. 2003, Ser. No. 60/550,784, filed Mar. 5, 2004, Ser. No. 60/591,668, filed on 28 Jul. 2004, and Ser. No. ______, filed Dec. 10, 2004, bearing attorney docket number 13154-002001, titled “Processing And Managing Genetic Information.”

### BRIEF DESCRIPTION OF THE DRAWINGS

FIG. 1 depicts a schematic of an exemplary gene sequencing workflow 100.

FIG. 2 depicts a schematic of an exemplary gene sequencing workflow 130 with setup and utility programs.

FIG. 3 depicts an exemplary process 200 for sequence data file screening.

FIG. 4 depicts exemplary representations of a plate map as a two-dimensional grid.

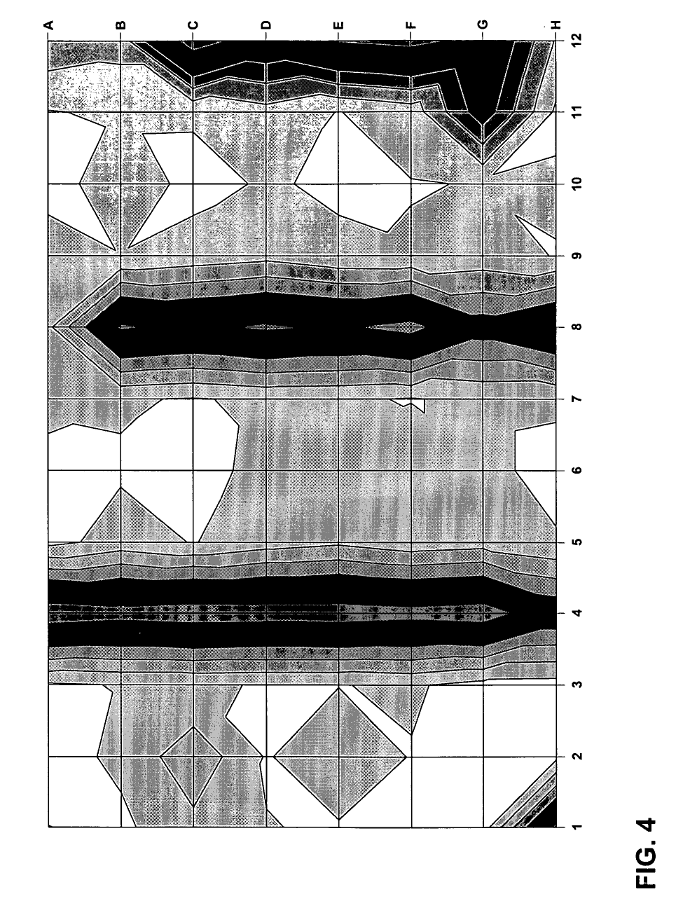

FIG. 5 depicts exemplary representations of a plate map as a three-dimensional graph.

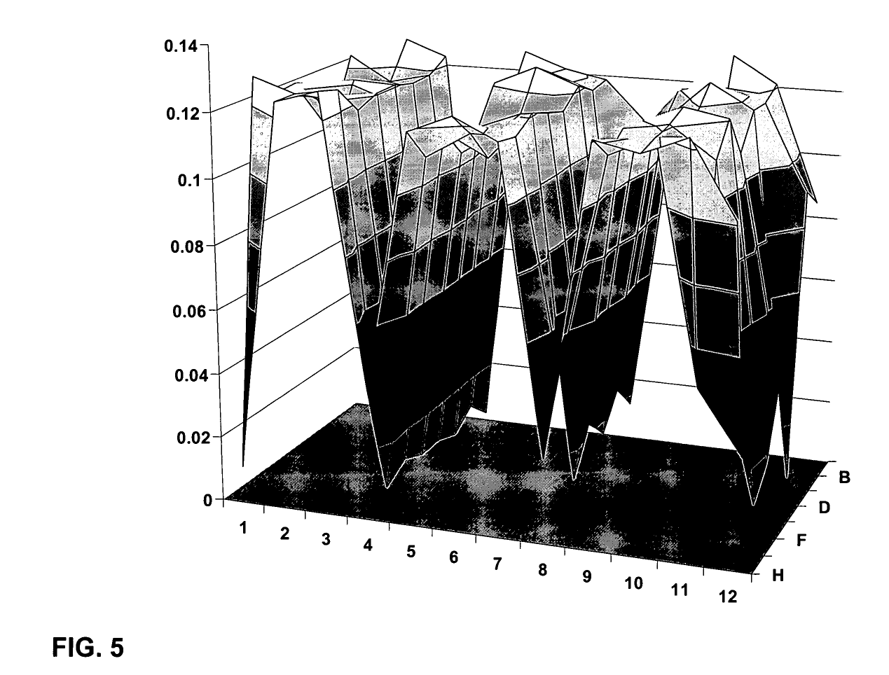

### DETAILED DESCRIPTION

1. Screening Tool

In one aspect, this disclosure features a screening tool (e.g., an automated screening tool) that can be used to avoid or minimize manual inspection of the sequence data for each amplicon that is analyzed. Sequence data is analyzed using one or more parameters; and in preferred embodiments a particular amplicon can be organized according to whether further review by a technician is needed. For example, sequence data for the amplicon can be identified, e.g., assigned a flag, indexed, or organized into a bin (e.g., a folder on a computer-based storage device). The identification can indicate a conclusion about the sequence, e.g., that it needs no manual review, that it needs manual review, and/or that it needs to be re-sequenced. Control sequences can be used and analyzed in the same manner. For example, every plate can include one or more control amplicons which can be used to determine if the plate, or specific amplicons on the plate, are acceptable or not.

Thus, an automated screening process has been developed that screens the processed amplicons to identify which need technician review, which can be automatically passed as normal, and which can be rejected as poor quality data which need resequencing.

This tool can also identify the type of review needed, e.g. review of low quality value base calls, review of potential sequence variants, and review of potential insertions or deletions in the sequence.

This tool reduces technician workload by eliminating the need to review data which is clearly normal and by eliminating the need to review data which is of such poor quality that it needs to be reprocessed. This tool also increases the efficiency of the technician review process by organizing the remaining amplicons by type of review needed. Because all of the amplicons passed on to the technician have at least one event (e.g., a base call) needing review, the possibility of a technician missing an event (e.g., a base call) which needs review is greatly reduced.

In one embodiment, this tool saves a list of the events which need review and uses this list to direct the technician to the relevant event. In one embodiment the tool not only directs the technician to the event, but actually presents the event to the technician for review. Both of these functions improve accuracy by eliminating the possibility of the technician overlooking an event which needs review.

1a. Identification of Amplicons which are Normal and Need No Further Review

An algorithm for identifying amplicons which are normal and need no further review has been developed. This algorithm, discussed in more detail below, uses preliminary base calling in combination with comparison to a reference sequence for this purpose. Examples of reference sequences include the sequence of a segment of a known gene or allele.

Preliminary base calling produces a call for each base and a quality value score derived from the probability of error in that base call. Typically, when a technician reviews each amplicon they use a limit criterion on the quality value score and review all base calls with quality value scores below the limit.

An exemplary screening algorithm, disclosed herein, automatically reads the results of the preliminary base calling and then compares the bases called to an appropriate reference sequence. In preferred embodiments, only the portion of the amplicon which is relevant to clinical evaluation is read or compared to the reference sequence and in some embodiments, only a portion of the amplicon is read or compared with the reference sequence. The portion can, e.g., include at least 5, 10, 20, or 100 nucleotides. In one embodiment, the portion is less than 90, 80, 70, 60, 50, 30% of the entire length of the amplicon.

The algorithm uses a preset limit criterion for the quality value score and identifies for each base call whether the call matches the reference sequence and whether the quality value score is above the limit criterion. Amplicons which have no variants from the reference sequence and for which all quality value scores are above the limit criterion are identified as normal and in need of no further review. In one embodiment, the algorithm automatically reads the preliminary base calling files, evaluates the amplicons, and marks the files as normal, as needing re-sequencing, or as needing further review, with regard to the correctness of the sequence determined. This marking can take any of many forms, in one embodiment the normal files are moved to a new directory, in another the names of the normal files are altered to identify them as normal, in another the files are added to a list which is presented to the technician or to a Laboratory Information Management System (LIMS).

Those skilled in the art understand that the calculation of a posterior probability of an hypothesis based on Bayesian inference includes (i) knowledge of events that have occurred (i.e. new evidence), and (ii) the probability of the hypothesis without knowledge of those events (i.e., the prior probability).

In one embodiment, the quality value scores are adjusted to account for Bayesian inference before they are compared to the limit criterion. In this case, new quality value scores are calculated from the posterior probability of error in the base calls, while the original quality value scores are the basis for the prior probability used in the Bayesian inference calculation. In one embodiment, the posterior probability is the probability of error in the base call given the “new evidence” that the base call matches the reference sequence. In another embodiment the posterior probability is the probability of error in the base call given that the base call is part of a characteristic sequence of base calls. The characteristic sequences have been, and are being, collected in a database to be used for estimating and evaluating base calls.

Bayesian inference can include more than one piece of new evidence. In one embodiment the posterior probability is the probability of error in the base call given that the base call matches a reference sequence and given that it is part of a characteristic sequence of base calls.

1b. Identification of Amplicons which Need to be Resequenced

An algorithm for identifying amplicons which need to be resequenced has been developed. This algorithm uses processing of the electropherogram to identify which amplicons need to be resequenced. In one embodiment it also uses preliminary base calling in combination with electropherogram signal characteristics for this purpose.

In one embodiment the electropherogram is processed in the following manner: The spectrum of the raw electropherogram is analyzed to identify its fundamental frequency. The electropherogram is essentially sinusoidal with multiple harmonics and sub-harmonics. The fundamental frequency in the electropherogram is the dominant frequency which is related to the presence of nucleotides in the amplicon. A band-pass filter which is configured to identify useful signals, e.g., one centered on the fundamental frequency, is used to identify useful signal as compared to noise. The portion of the electropherogram signal which is passed by the filter is considered to be signal and that which is not passed is considered to be noise. The ratio of signal to noise can be used as a measure of the quality of the electropherogram. A measure of amplitude (in one embodiment, the average amplitude) of the electropherogram signal can also be measured. One measure of the average amplitude is the standard deviation of the electropherogram. The measure of amplitude can be used individually or in combination with signal to noise ratio as a measure of the quality of the electropherogram.

These two electropherogram characteristics, amplitude and signal-to-noise ratio, can be used either individually or together to identify amplicons which need to be re-sequenced. Amplicons with amplitude below a given cutoff level and/or with signal to noise ratios below a given cutoff level are considered to be of such low quality that they need to be re-sequenced. The cutoff criteria can be established to suit the needs of the user.

Most amplicons include low quality signal at their beginning and end. These leading and trailing portions of the amplicon are not included in base calling or analysis. In one embodiment, these leading and trailing portions of the amplicon are not included in the amplitude and signal-to-noise measurements so that analysis and results based on these measures better represent the portion of the amplicon which is actually used in base calling.

Preliminary base calling produces a processed electropherogram. The algorithm described above can be applied to this processed electropherogram, just as it was applied in the above description to the raw electropherogram.

The electropherogram is usually represented as four separate signals, one for each base nucleotide, A, G, C, and T. These four signals can be added together and processed as a one continuous electropherogram signal. The processing as described above can be applied to either the individual signals or to the combined signal.

In another embodiment, the amplicons which are candidates for resequencing are subject to evaluation of preliminary base calling. The amplicons are subject to re-sequencing only if the preliminary base calls indicate that the value for a preselected parameter, e.g., the mean probability of error in base calling, is higher than established cutoff criteria. The cutoff criteria can be set to suit the needs of the user.

The two approaches described, the one using electropherogram characteristics and the other using preliminary base calling characteristics, can be used independently or in conjunction to provide a final determination as to whether an amplicon should be re-sequenced.

1c. Identification of Amplicons which Potentially have Insertions or Deletions in their Sequence.

An algorithm has been developed to distinguish between two classes of amplicons, one of which includes amplicons of low quality (in some embodiments these amplicons are resequenced or identified as being in need of resequencing), and the second which includes amplicons with numerous heterozygous base calls resulting from insertions and/or deletions in the sequence. This algorithm uses processing of the electropherogram to identify to which of these two classes an amplicon belongs. In one embodiment it also uses preliminary base calling in combination with electropherogram signal characteristics for this class identification.

In one embodiment the electropherogram is processed in the following manner:

The spectrum of the raw electropherogram is analyzed to identify its fundamental frequency.

A band-pass filter which is configured to identify useful signals, e.g., one centered on the fundamental frequency, is used to identify useful signal as compared to noise. The portion of the electropherogram which is passed by the filter is considered to be signal and that which is not passed is considered to be noise. The ratio of signal to noise can be used as a measure of the quality of the electropherogram.

The measure of amplitude, e.g., average amplitude, of the electropherogram signal is also measured. One measure of this average amplitude is the standard deviation of the electropherogram. The measure of amplitude can be used individually or in combination other information, e.g., with signal to noise ratio as a measure of the quality of the electropherogram.

As discussed elsewhere herein, most amplicons include low quality signal at their beginning and end. In some embodiments these leading and trailing portions of the amplicon are not included in base calling or analysis. In one embodiment, these leading and trailing portions of the amplicon are not included in the amplitude and signal-to-noise measurements so that analysis and results based on these measures better represent the portion of the amplicon which is actually used in base calling.

These two electropherogram characteristics, amplitude and signal-to-noise ratio, can be used either individually or together to classify the quality of the electropherogram. A high quality electropherogram which has a large number of variants in its preliminary base call is identified as a probable candidate for having insertions and/or deletions in its sequence.

Preliminary base calling produces a processed electropherogram. The class identification algorithm described above can be applied to this processed electropherogram, just as it was applied in the above description to the raw electropherogram.

The electropherogram can include representations for four separate signals, one for each base nucleotide (e.g., A, G, C, and T). These four signals can be combined into a single signal and processed as a one continuous electropherogram signal. The processing as described above can be applied to either the individual signals or to the combined signal.

A high quality electropherogram which has a relatively large number of variants (generally adjacent to one another) in its preliminary base call can be identified as a probable candidate for having insertions and/or deletions in its sequence.

Amplicons of good quality which have a heterozygous insertion or deletion in their nucleotide sequence can look similar to amplicons of poor quality in that both types of amplicon have a large number of low quality value base calls and a large number of sequence variants. The distinction between these types of amplicons is in the quality of the electropherogram, and in the distribution of low quality and variant calls. A homozygous insertion or deletion can exhibit normal quality values, but a large number of sequence variants.

2. Use of Improved Base Calling in a Screening Tool and Secondary Processing Tool.

This section describes, inter alia, an algorithm that improves the accuracy of the estimate of the probability of error in each base call. In base calling, the term quality value refers to a quantity calculated from this estimate of the probability of error in a base call. Many base calling algorithms produce a quality value which is based on characteristics of the electropherogram.

When an amplicon is sequenced to identify variations from a reference sequence, the information in the reference sequence can be used to improve the accuracy of the quality value associated with each base call. This can be done by using, e.g., one or more of the following approaches.

2a. First, the quality value scores can be calculated to reflect the fact that the base calls of interest are only those in the region of the amplicon which correspond to the reference sequence. This region of the amplicon typically has a very high quality signal. Quality value scores produced by preliminary base calling programs are typically based on the entire amplicon. The probabilities associated with those base calls may not be properly represented for the region under consideration.

In one embodiment, the algorithm described herein calculates quality values based on the fact that the base calling is occurring in the region of the amplicon which corresponds to the reference sequence.

2b. Second, the base calls can be compared to the known reference sequence. The total population of base calls can be separated into those which match the reference sequence and those which do not. Methods disclosed herein can consider these two populations separately in calculating quality value scores.

2c. Third, the base calls can be compared to known signature sequences. Specific sequences of bases have consistent signatures, which may include low amplitudes or low quality values for specific bases within the signature. The algorithm calculates the quality value in consideration of the fact that a particular call is part of a specific signature. The signature sequence comes from a library of signature sequences. This signature technique can also be applied in the absence of a specific reference sequence.

A signature sequence is a series of nucleotides associated with a value for a selected parameter for one of the nucleotides in the signature. It gives a value for a particular base within a particular context, e.g., a particular sequence context. E.g., base X4 may give a particular value, e.g., a quality value, an amplitude, or other value, when found in the context of the sequence X1-X2-X3-X4. For example, the apparent quality value of X4 could be lower in this context than in other contexts, e.g., in signature X5-X6-X7-X4 or signature X1-X6-X4-X8. If X4 is found in this context, in a particular signature, in the amplicon, then a value which might otherwise not meet a selection criterion would still be acceptable and the identity accepted without resequencing or without further review, e.g., of the raw or processed electropherogram. Thus, upon reviewing a base call with a given value, e.g., a quality value, one uses signature analysis as an indication of the correctness of the call. The value for a given position can be compared to a library of signatures. The signatures can be, e.g., 3, 4, 5-10 bases in length. A library can include signatures which encompass some, many, or all (e.g., 80, 90, 95%, or all) possible combinations, For example, if all possible combinations are used, and fragments of 5 nucleotides are used, the library would have 1024 signatures.

These techniques and other related techniques can use Bayesian probability estimates. The techniques calculate a quality value given new evidence. In the first case the new evidence is the fact the sequence is in the region of the reference sequence. In the second case the new evidence is that the base call matches the base call from the reference sequence. In the third case, the new evidence is that the base call matches a known signature sequence. Other cases of new evidence can also be used.

Better accuracy in identification of the probabilities associated with base calls reduces the need for technician review, and in combination with the screening tool presented herein will increase the number of amplicons which can be eliminated from the technician workflow. The use of signature identification can be effective for de novo sequencing as well as reference based sequencing and may cover 70-80% of the review events.

3. Sequencer Function Monitoring Tool

Also provided herein is a method and algorithm to track and analyze the functioning of nucleic acid sequencing apparati, particularly automated DNA sequencers. This algorithm can be incorporated in a tool which identifies deviations in performance, e.g., diminished function. The tool can produce a signal upon identification of a deviation and can, e.g., produce an alert, e.g., for the operator of the sequencer. The signal or alert can indicate that a problem exists and can recommend corrective action.

A typical automated sequencer uses one or more platforms, e.g., plates, containing many reaction chambers, e.g., wells, or tubes, which hold the samples to be analyzed. A plate map is used to map each DNA sequence to the sample from which it was derived.

In one implementation, the characteristics of each amplicon are identified by a preliminary base calling program and can also be calculated by a screening tool and secondary processor tool. These characteristics are mapped to the plate and well from which the amplicon is derived. This mapping can identify systematic problems within each sequencing run, and also allows a comparison of maps from plate to plate, run to run, day to day, and week to week, to identify problems which may be developing in the DNA sequencer or in upstream liquid handling systems or in reagents.

The map of characteristics to the plate can be depicted in a variety of forms, most typically as a two-dimensional map that corresponds to the plate design. Characteristics can be represented, e.g., using a color scale, contours, or by graphing along a third dimension or by an identifier associated with a particular characteristic. However, there is no need for the tool to generate a depiction or display of the plate map. The tool can itself process the map of characteristics to determine if there is a pattern of altered performance, e.g., associated with a component of the sequencer. Based on the pattern, the tool can also identify the deviant component or suggest possible components for inspection. Exemplary components which can be identified as have altered performance include fluorescence detectors, capillaries, pipettes, reagent reservoirs, and so forth.

Sometimes the attempt to sequence the DNA samples simply fails, and these failures can be a clear indication of sequencer malfunction. The algorithm can identify these failed tests, but also can be a sensitive means for identifying problems before the point of sequencing failures. For example, sequence data characterized by consistently low amplitude signal can still have high quality value scores and may be processed without difficulty. However, such data may be indicative of a deteriorating situation which may eventually lead to failure to read the sequence of a sample. Thus, the sequencer function monitoring tool can not only provide a way of monitoring sequencer performance but can also provide a way of evaluating a base call or quality value and determining whether a call should be accepted, reviewed or resequenced.

By identifying problems before they lead to wide scale failures, the monitoring tool enables more efficient use of automated sequencers and leads to a lower overall failure rate in high-throughput DNA sequencing. Furthermore, samples which are sequenced in a sub-optimal fashion often have a high number of inaccurate or ambiguous base calls. Keeping the sequencer functioning in optimal fashion reduces base calling errors and the time required for reviewing and editing the base calls.

Automated DNA sequencers process samples plate by plate, and can be loaded with a number of plates, each of which will be processed automatically in turn. The monitoring tool tracks sequencer function plate by plate. In one embodiment, the tool includes a notification function so that when a problem is identified, the sequencer operator is notified and can intervene if necessary. The notification allows the operator to interrupt the processing of a group of plates and make any necessary adjustments, rather than allowing all the plates in the group to be processed in an inappropriate or sub-optimal fashion.

The notification function can take any of a number of forms, including a message on the screen of the DNA sequencer, a message transmitted to the screen of other designated computers connected via internet, local area network, wireless network or other technology used for computer-to-computer communication, an email message, a message transmitted using instant messaging technology, a message transmitted to a telephone, personal digital assistant, or other personal communication device, and a message transmitted by any means to the sequencer operator. The term message includes all types of communication including, e.g., text, audio, and graphical.

In one embodiment, the monitoring tool recommends corrective actions in addition to producing a notification for the operator regarding malfunction. The tool is able to do this by relating sequencer malfunction to a knowledge-base of corrective actions. There are multiple sources for such a knowledge-base. The knowledge-base can be either individually or in combination, derived from, or a link to, the sequencer manufacturer's published trouble shooting recommendations, developed from an operator's own experience with sequencer malfunctions, and developed from the shared experience of users of the monitoring tool, e.g., using information shared on an internal or external computer network.

In one embodiment, the amplicons are characterized according to a measure of the amplitude of the raw electropherogram and signal to noise ratio of the raw electropherogram as discussed above.

As demonstrated in test data, when the locations of amplicons with low quality signals are highly correlated, rather than being randomly distributed., the correlation can indicate progressively reduced functionality of specific parts of the process, such as deteriorating capillaries, degradation of reagents, partially blocked or malfunctioning pipettes, and vacuum or heating problems.

The specifics of the type of amplicon characteristic and distribution of the amplicon characteristic can be used to identify the nature and location of problems developing in the sequencer.

4. Base Calling

This section describes an embodiment of a method disclosed herein. An automated pattern recognition strategy, e.g., one which uses prior knowledge of the correct DNA sequence, would have advantages over an approach in which any nucleotide might appear at any position.

The pattern of nucleotide signals in a known DNA sequence is used to compare with that of a test sequence. Two embodiments of pattern recognition include:
1) using a known DNA sequence (e.g., a sequence of the normal or wild-type gene) as the basis for comparison, and “training” the base calling program to a specific pattern, within a window of nucleotides of a given width, to acknowledge the importance of the immediate environment surrounding a given base to the appearance of that base in a chromatogram.

2) using a library of small (3, 4, 5-10 base) fragments of known DNA sequence (DNA fragment standards, DFS) which encompass some, many, or all (e.g., 80, 90, 95%, or all) possible combinations, as the basis with which to read a test sequence. For example, if all possible combinations are used, and fragments of 5 nucleotides are used, the library would have 1024 DFSs. DFSs can be obtained, e.g., from pre-existing DNA sequences residing in DNA sequence repositories or generated de novo. For each unique DFS, the analysis of multiple examples is used to build a refined pattern, e.g., a pattern including or based on averages, and ranges, of sequence appearance.

In either case, the resulting reading of the test sequence can be used to further train the reading program for the interpretation of subsequent test sequences. For example, the sequence is modeled using a Markov approach.

Frequently the trace for a given nucleotide is influenced by the several (e.g., about four) bases that come before it. The trace can also be influenced by downstream bases within the template (e.g., the sequencing reaction, e.g., a polymerase component may “see” these downstream bases, or the higher order structure of the template downstream of the growing polymer may influence its growth).

The prediction method can account for sequencing rules, such as:
C's after T's are usually small
If there is more than one G after an A, the first G is small.
If there is more than one C after a G, the first C is small.
Sometimes in a string of 4 G's, the 2nd or 3rd G is small.
T's after G's are usually small.
In a string of 4 or more A's, the second A is usually small.

DFSs could be generated in plasmid vectors, and be sequenced. Alternatively, DNA sequence information in existing repositories, either diagnostic DNA sequencing centers or academic or commercial sequencing laboratories can be analyzed.

The size of the critical region used for DFS can be varied, e.g., to find a size which returns accurate reads, e.g., using a test set of sequence traces. The method can be used to generate patterns that are gene- and/or position-independent, e.g., with respect to terminal nucleotide appearance.

Patterns can be generated by data mining a large repository of DNA sequence information to establish the correct pattern rules. The repository can employ the same DNA sequencing chemistry and DNA sequencing machines as will be used in future sequencing, as the patterns will likely be dependent upon both the chemistry and the machinery. In other words, patterns can be developed that are chemistry and/or machine specific. Other patterns may be general.

The patterns and rules can be used to evaluate (e.g., detect) the presence of heterozygous DNA bases at a given nucleotide position, by systematically introducing heterozygous nucleotides at each terminating position and analyzing the pattern. In one embodiment, Markov methods (e.g., hidden Markov models) are used for pattern recognition. In another embodiment, the program is trained, e.g., using a Bayesian model.

Computer Implementations

The invention can be implemented in digital electronic circuitry, or in computer hardware, firmware, software, or in combinations thereof. Methods of the invention can be implemented using a computer program product tangibly embodied in a machine-readable storage device for execution by a programmable processor; and method actions can be performed by a programmable processor executing a program of instructions to perform functions of the invention by operating on input data and generating output. For example, the invention can be implemented advantageously in one or more computer programs that are executable on a programmable system including at least one programmable processor coupled to receive data and instructions from, and to transmit data and instructions to, a data storage system, at least one input device, and at least one output device.

Each computer program can be implemented in a high-level procedural or object oriented programming language, or in assembly or machine language if desired; and in any case, the language can be a compiled or interpreted language. Suitable processors include, by way of example, both general and special purpose microprocessors. A processor can receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks such as, internal hard disks and removable disks; magneto-optical disks; and CD_ROM disks. Any of the foregoing can be supplemented by, or incorporated in, ASICs (application-specific integrated circuits).

An example of one such type of system includes a processor, a random access memory (RAM), a program memory (for example, a writable read-only memory (ROM) such as a flash ROM), a hard drive controller, and an input/output (I/O) controller coupled by a processor (CPU) bus. The system can be preprogrammed, in ROM, for example, or it can be programmed (and reprogrammed) by loading a program from another source (for example, from a floppy disk, a CD-ROM, or another computer).

The hard drive controller is coupled to a hard disk suitable for storing executable computer programs, including programs embodying the present invention, and data including storage. The I/O controller is coupled by means of an I/O bus to an I/O interface. The I/O interface receives and transmits data in analog or digital form over communication links such as a serial link, local area network, wireless link, and parallel link.

One non-limiting example of an execution environment includes computers running LINUX RED HAT® OS, WINDOWS® XP or NT 4.0 (Microsoft) or better or SOLARIS® 2.6 or better (Sun Microsystems) operating systems. Browsers can be MICROSOFT INTERNET EXPLORER® version 4.0 or greater or NETSCAPE NAVIGATOR® version 4.0 or greater. Computers for databases and administration servers can include WINDOWS® NT 4.0 with a 400 MHz PENTIUM® II (Intel) processor or equivalent using 256 MB memory and 9 GB SCSI drive. For example, a SOLARIS® 2.6 Ultra 10 (400 Mhz) with 256 MB memory and 9 GB SCSI drive can be used. Other environments can also be used.

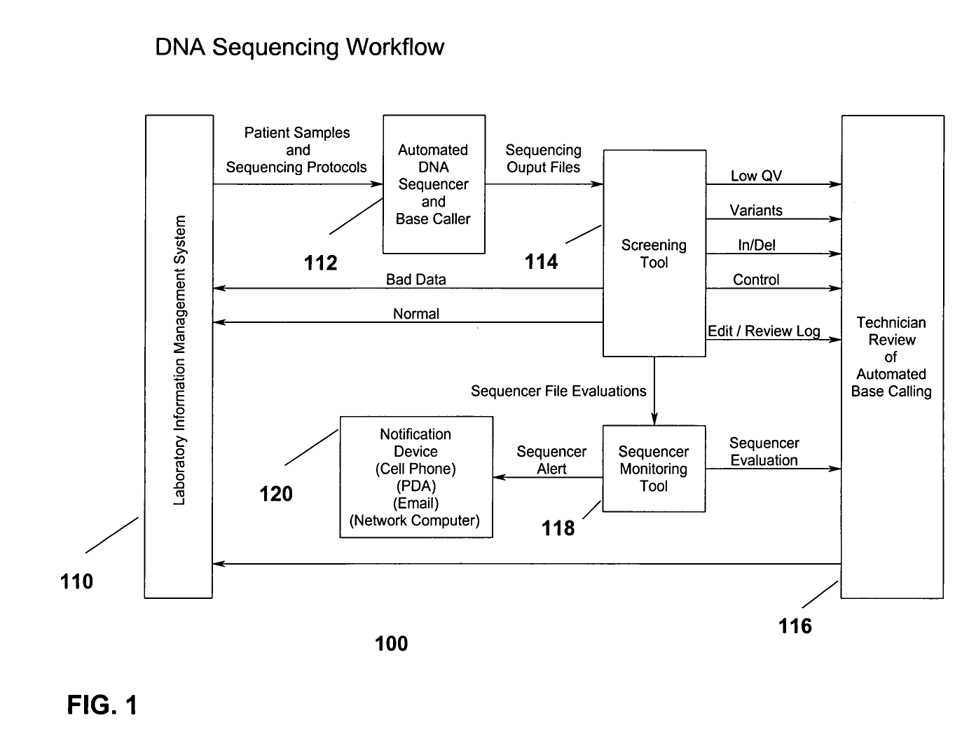

In one exemplary implementation 100 illustrated in FIG. 1, a LIMS 110 provides patient samples and sequencing protocols. These are used by an automated DNA sequencer and base caller 112 to generate sequencing output files for a screening tool 114. The screening tool 114 can evaluate the output files and route indications of bad data and normal data to the LIMS 110. The screening tool 114 can also trigger technician review 116, e.g., for files with a low QV score, variants, IN/DELs, and control files. The screening tool 114 can also generate and send to the technician 116 a log of events (e.g., potential edits and/or reviews). Information from the screening tool can also be passed to the sequencer monitoring tool 118. The sequencer monitoring tool 118 can detect potential performance aberrations and provide a sequencer alert by triggering a notification device 120 or by sending information for technician review 116.

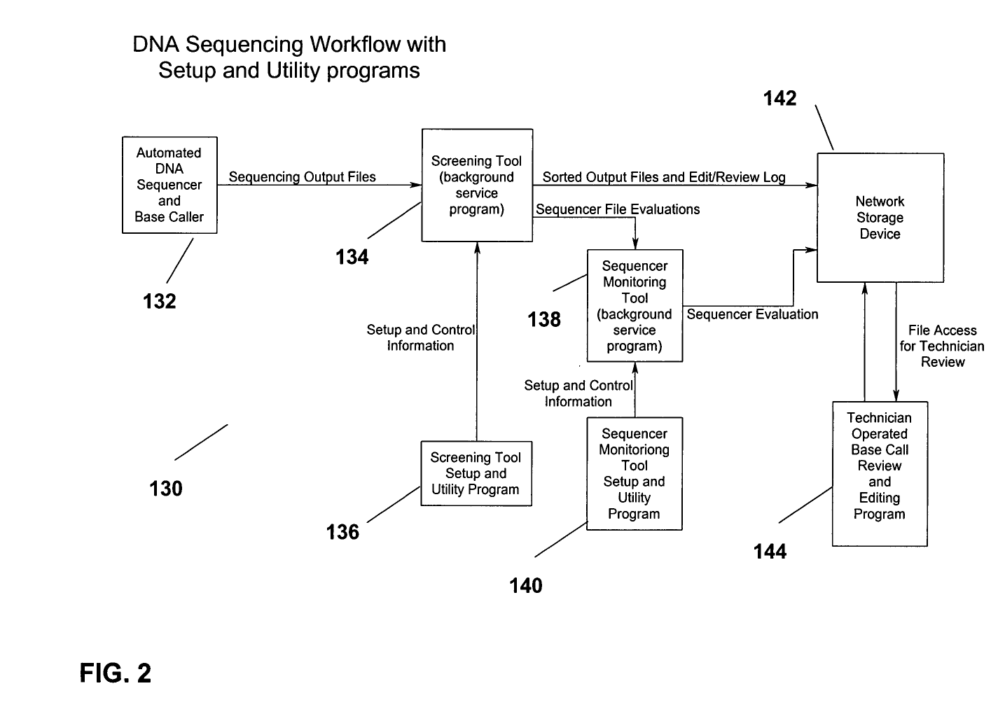

In the exemplary workflow 130, illustrated in FIG. 2, an automated DNA sequencer and base caller 132 routes sequencing output files to a screening tool 134, which can, for example, run as a background service program. The operation of the screening tool 134 can be controlled, e.g., by a screening tool setup and utility program 136. The screening tool 134 can sort output files and can generate an Edit/Review log, e.g., for a network storage device 142. The network storage device can be accessed for technician review, e.g., using a technician-operated base call review and editing program 144 which modifies files and logs. The screening tool 134 can also provide sequencer file evaluations which are processed by a sequencer monitoring tool 138 (which also can run as a background service program). The sequencer monitoring tool setup and utility program 140 can communicate setup and control information to the sequencer monitoring tool 138.

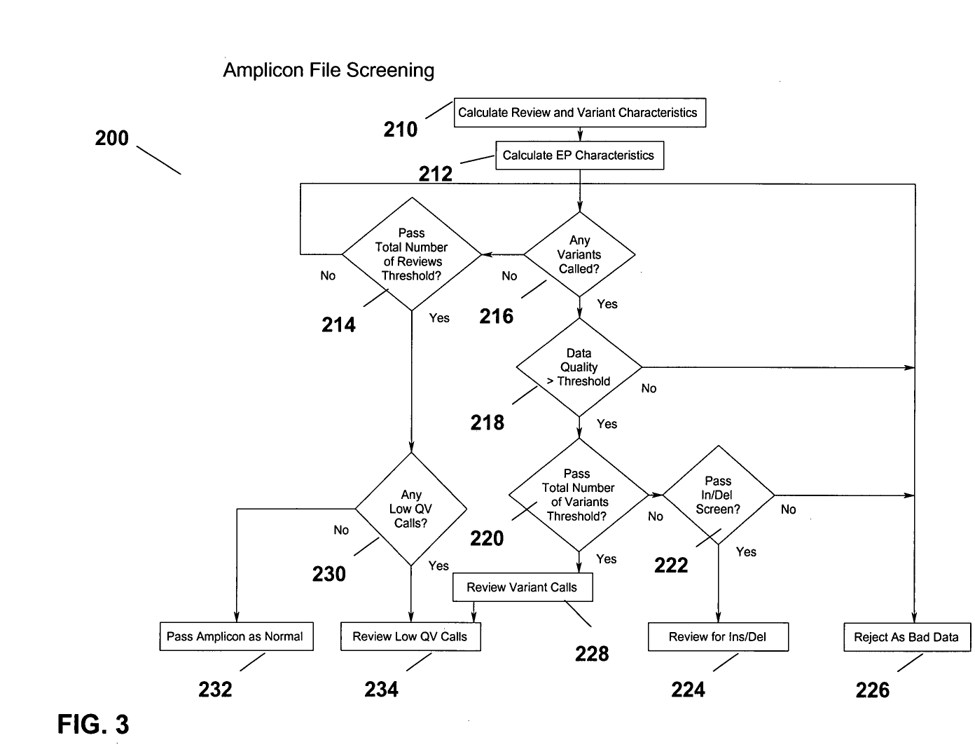

FIG. 3 provides an exemplary process for amplicon file screening. The process includes calculating 210 review and variant characteristics and calculating 212 electropherogram (EP) characteristics. A file is evaluated to determine if they have any variants called 216. If not, a file is evaluated to determine if they pass the total number of “reviews” threshold 214. Here a “review” indicates a flag requiring technician review. If it does not pass the threshold, it can be rejected as bad data 226. If it does pass the threshold and has no low quality value calls 230, the file can be indicated as normal 232. If it does have low quality value calls 230, it can be indicated for review of low quality value calls 234.

If a variant is called, it is evaluated for data quality 218. If the data quality is less than a threshold, the file can be rejected as bad data 226. If the data quality is greater than a threshold, the file can be evaluated to see if it passes the total number of variants threshold 220. If it does, it can be reviewed for variant calls 228. If it does not, it can be screened 222 for IN/DELs. If IN/DELS are detected, it can be indicated for IN/DEL review 224, otherwise it can be indicated as bad data 226.

Applications

The methods described herein can be used in a variety of applications. The methods can be used to process sequence data for a sequence for which there is a known reference sequence or for “de novo” sequencing of sequence without reference to or knowledge of a reference sequence. For example, a method can be applied to a known gene in an individual and also to process sequence data for an unknown gene (e.g., a novel gene). For example, they can be used to process sequence data for (i) diagnostic sequencing of human genes, e.g., to provide patient diagnostics based on genes associated with human disorders; (ii) diagnostic sequence of non-human genes (e.g., genes of non-human animals of veterinary interest and genes of bacterial, viral or parasitic organisms, e.g., pathogenic or commensal organisms.). The methods can be used to evaluate sequence data from genome sequence projects. The genomes of numerous organisms are being sequenced. These organisms include pathogens, mammalians, and organisms of environmental interest. The genomes of human individuals are also being sequenced, e.g., to obtain better maps of variants and for epidemiology. Methods described herein can also be applied to other sequences, e.g., sequencing to confirm the sequence of an engineered or synthetic construct, samples from food, agricultural, or forensic samples.

### EXAMPLE 1

### Base Calling Results

Sequence data for 264 amplicons were obtained. This data include a total of 54,234 bases called. 4.3% of the calls needed review. Total edits would be <0.043%. After automated processing of the sequence data for each of the amplicons, 136 of the 264 (51.5%) needed no manual review.

60 amplicons (22.7%) needed only one review. By adjusting the quality value scores to account for the posterior probability of a match to the reference sequence, the number of amplicons requiring no manual review was increased to 78%.

Other embodiments are within the scope of the following claims.

1. A method of processing sequence data, the method comprising:
obtaining sequence data that comprises nucleotide assignments for positions in a sequence and performance characteristics; and
automatically sorting the sequence data into categories based on necessity for further review of the correctness of the sequence, wherein the categories include:
(i) one or more categories for sequence data that do not require further review of the correctness of the sequence; and
(ii) one or more categories for sequence data that require further review of the correctness of the sequence.

2. The method of claim 1 wherein the categories (i) of sequence data that do not require further review of the correctness of the sequence comprise a category for sequence data that includes accepted performance characteristics and nucleotide assignments that match a reference sequence

3. The method of claim 1 wherein the categories (i) of sequence data that do not require further review of the correctness of the sequence comprise a category for sequence that includes a threshold number of unaccepted performance characteristics and at least a threshold number of nucleotide assignments that do not match a reference sequence.

4. The method of claim 1 wherein the categories (i) of sequence data that do not require further review of the correctness of the sequence comprise a category for sequence data that includes at least one unaccepted performance characteristic at a position, which characteristic is predicted to occur within the context of the position.

5. The method of claim 1 wherein the categories (ii) that do require further review of the correctness of the sequence comprise a category for sequence data that includes at least a threshold number of nucleotide assignments that do not match a reference sequence and a threshold number of accepted performance characteristics.

6. The method of claim 1 wherein the categories (ii) that do require further review of the correctness of the sequence comprise a category for sequence data that includes a nucleotide assignment that does not match a reference sequence and an accepted performance characteristic at the position corresponding to the mismatch.

7. The method of claim 6, further comprising associating an identifier which indicates there is a need for review of the sequence.

8. The method of claim 1 wherein the sequence data is pre-processed by software that determines nucleotide assignments and quality values.

9. The method of claim 1 wherein the performance characteristics comprise quality value scores for positions in the sequence.

10. The method of claim 1 wherein the performance characteristics comprise amplitudes and/or peak widths for positions in the sequence.

11. The method of claim 1 wherein multiple files comprising sequence data are handled, and the files are organized by the automatic sorting.

12. A method of processing sequence data, the method comprising:
obtaining sequence data that comprises nucleotide assignments for positions in a sequence and performance characteristics; and
evaluating the sequence data by determining one or more of the following:
(i) if the sequence data includes accepted performance characteristics and nucleotide assignments that match a reference sequence;
(ii) if the sequence data includes a threshold number of unaccepted performance characteristics and at least a threshold number of nucleotide assignments that do not match a reference sequence;
(iii) if the sequence data includes at least one unaccepted performance characteristic at a position, which characteristic is predicted to occur within the context of the position;
(iv) if the sequence data includes at least one unaccepted performance characteristic at a position, which characteristic is accepted based on a revised quality value score;
(v) if the sequence data includes at least one unaccepted performance characteristic at a position and nucleotide assignments that match a reference sequence;
(vi) if the sequence data includes at least a threshold number of nucleotide assignments that do not match a reference sequence and a threshold number of accepted performance characteristics; and/or
(vii) if the sequence data includes a nucleotide assignment that does not match a reference sequence and an accepted performance characteristic at the position corresponding to the mismatch.

13. The method of claim 12 wherein (iv) is determined using a Bayesian inference.

14. The method of claim 12 wherein the inference is determined using two populations.

15. The method of claim 12 wherein the sequence data is evaluated for at least two of the seven characteristics of (i)—(vii).

16. The method of claim 12 wherein the sequence data is evaluated for all seven characteristics of (i)—(vii).

17. The method of claim 12 wherein the sequence data is indicated for operator review if it has characteristic (v), (vi) or (vii).

18. A dataserver comprising storage having encoded therein multiple files of sequence data that comprises nucleotide assignments for positions in a sequence and performance characteristics, wherein the files are organized according to one or more of the following categories, in which the sequence data:
(i) includes accepted performance characteristics and nucleotide assignments that match a reference sequence;
(ii) includes a threshold number of unaccepted performance characteristics and at least a threshold number of nucleotide assignments that do not match a reference sequence;
(iii) includes at least one unaccepted performance characteristic at a position, which characteristic is predicted to occur within the context of the position;
(iv) includes at least one unaccepted performance characteristic at a position, which characteristic is accepted based on a revised quality value score;
(v) if the sequence data includes at least one unaccepted performance characteristic at a position and nucleotide assignments that match a reference sequence;
(vi) includes at least a threshold number of nucleotide assignments that do not match a reference sequence and a threshold number of accepted performance characteristics; and/or
(vii) includes a nucleotide assignment that does not match a reference sequence and an accepted performance characteristic at the position corresponding to the mismatch.

19. A method of identify insertions or deletions in sequence data, the method comprising:
obtaining sequence data that comprises nucleotide assignments for positions in a sequence and performance characteristics; and
evaluating if the sequence data includes at least a threshold number of nucleotide assignments that do not match a reference sequence and a threshold number of accepted performance characteristics.

20. The method of claim 19 further comprising adding or subtracting signals expected for a normal sequence from a region that includes mismatches to the reference sequence, and determining if the remaining signal corresponds to the reference sequence shifted by one or more positions.

21. A method for evaluating sequence data, the method comprising:
identifying at least one position in a sequence that has an unaccepted performance characteristic; and
determining if the unaccepted performance is predicted to occur within the context of the position.

22. The method of claim 21 wherein the step of determining comprises accessing a database that comprises records that associates performance characteristics and sequence information.

23. The method of claim 22 wherein the database comprises records for all possible 3-mer, 4-mers, or 5-mers.

24. The method of claim 22 wherein the database comprises records for at least 10% of all possible 4-mers.

25. The method of claim 22 wherein the database is generated by evaluating sequence data produced from different samples, and recurring patterns of performance characteristics associated with a particular context of nucleotides are stored in the database.

26. The method of claim 21 further comprising indicating the sequence data as accepted if the unaccepted performance is predicted to occur within the context of the position.

27. The method of claim 21 wherein the unaccepted performance comprises a quality value less than a threshold.

28. A method for evaluating sequence data, the method comprising:
providing a database which includes sequences and sets of values associated with the respective sequences, the values being a value for a performance characteristic; and
locating at least one position in a sequence, which is a position subject question, and at least one additional position; and
determining if the nucleotide assignment for a position and the at least one additional position of a set of positions and their corresponding values match a record in the database.

29. The method of claim 28 further comprising providing an indication that sequence data should be retained, if a match is detected.

30. A method for evaluating sequence data, the method comprising:
receiving sequence data that comprises nucleotide assignments for positions in a sequence and values for a parameter that characterizes each position;
evaluating the sequence data to identify a position, if any, for which the value is indicated as deviating from normal;
comparing a pattern of values at consecutive positions, one of which is the identified position, to a database that associates patterns of values with strings of nucleotide assignments; and
indicating the sequence data as accepted if the pattern of values for the consecutive positions is indicated by the database as associated with the nucleotide assignments for the consecutive positions.

31. A computer database that stores records that associate performance characteristics for a string of nucleotide assignments.

32. The database of claim 31 wherein the database comprises records for all possible 3-mer, 4-mers, or 5-mers.

33. The database of claim 31 wherein the database comprises records for at least 10% of all possible 4-mers.

34. The database of claim 31 wherein the performance characteristics correspond to one or more of: quality values, scaled amplitudes, peak widths, or amplitude/peak width ratios, and values that are functions of these characteristics.

35. A method for evaluating the performance quality of one or more datasources for nucleic acid sequence data, the method comprising:
providing values for one or more parameters obtained from sequence data output from multiple datasources,
organizing the parameter values according to datasource, and
identifying, from the organized parameters, an indication of performance quality of one or more of the datasources or a component associated with the datasources.

36. The method of claim 35 wherein the multiple datasources correspond to individual reaction chambers in a nucleic acid sequence apparatus.

37. The method of claim 35 wherein the multiple datasources correspond to capillaries located in parallel in an automated nucleic acid sequencer.

38. The method of claim 35 wherein the step of organizing and/or identifying comprises organizing the parameters as a data structure comprising two dimensions.

39. The method of claim 38 wherein the data structure corresponds to a plate map.

40. The method of claim 38 wherein the step of organizing and/or identifying comprises displaying information in a two dimensional grid, wherein parameters obtained from the same datasource are represented at positions along a line on one of the dimensions of the grid.

41. The method of claim 35 wherein the step of organizing and/or identifying comprises detecting patterns indicative of reduced performance of one or more of the datasources.

42. The method of claim 41 wherein detection of a pattern indicative of reduced performance triggers an alert to a user.

43. The method of claim 41 wherein detection of a pattern indicative of reduced performance triggers a flag that arrests the sequencer from processing another plate or sample.
